(19) United States
(12) Patent Application Publication
(10) Pub. No.: US 2024/0189429 A1
(43) Pub. Date: Jun. 13, 2024

(54) METHOD FOR ANCHORING AND MODIFYING NANO-DRUG ON SURFACE OF LIVING CELL

(71) Applicant: CHINA PHARMACEUTICAL UNIVERSITY, Nanjing, Jiangsu (CN)

(72) Inventors: Can ZHANG, Nanjing (CN); Caoyun JU, Nanjing (CN); Meixi HAO, Nanjing (CN); Siyuan HOU, Nanjing (CN)

(73) Assignee: CHINA PHARMACEUTICAL UNIVERSITY, Nanjing, Jiangsu (CN)

(21) Appl. No.: 17/773,492

(22) PCT Filed: Oct. 10, 2020
(86) PCT No.: PCT/CN2020/120073

§ 371 Date: Apr. 29, 2022

(30) Foreign Application Priority Data
Oct. 30, 2019 (CN) 201911047108.5
Aug. 14, 2020 (CN) 202010815772.6

(51) Int. Cl. A61K 47/54; A61K 47/60

(52) U.S. Cl. CPC A61K 47/543; A61K 47/544; A61K 47/60

(57) ABSTRACT

A method for anchoring and modifying a nano-drug on the surface of a living cell includes: introducing an active reactive group to a living cell surface by a hydrophobic tail chain of a cell membrane anchoring molecule; modifying the nano-drug surface with a corresponding reactive group; and executing a biological orthogonal click reaction on the active reactive group modified on the living cell surface and the corresponding reactive group modified on the nano-drug surfae, to anchor and modify the nano-drug to the cell surface obtaining a living cell modified with the nano-drug. The method provides a new technical platform for cell modification and has very wide application prospects. Compared with pure cells and pure nano-drugs, a cell drug obtained by the above-mentioned cell modification technology has an optimal treatment effect and provides a new idea and a new drug for treating various diseases.

### TECHNICAL FIELD

The present invention belongs to the field of biotechnology, and in particular relates to a method for anchoring and modifying a nano-drug on the surface of a living cell.

### BACKGROUND

With the development of nanotechnology, the application of nano-drugs in the treatment of various diseases has become more and more extensive. Since the advent of the first nano-drug in 1964, other types of nano-drugs, such as polymeric micelles and albumin nanoparticles, have come out one after another. So far, 36 nano-drugs have come on the market. However, nano-drugs have certain limitations. From a drug delivery site to a target site, several physiological barriers need to be overcome, including blood, tissues, cells, etc. The amount of drug that finally reaches the target site is only 5%-8% of the administered dose, the targeting efficiency is low, and the clinical efficacy is not ideal.

To improve the targeting efficiency of nano-drugs, application of endogenous cells as a tool to deliver the nano-drugs has been extensively studied. On the one hand, endogenous cells can help nano-drugs escape recognition of the Reticuloendothelial System (RES), and improve the ability of nano-drugs to enrich in specific tissues, thereby improving their in vivo residence time and targeting efficiency. On the other hand, endogenous cells such as T cells (chimeric antigen receptor T cells (CAR-T cells), T cell receptor-gene engineered T cell (TCR-T cells)), and Natural killer cells (NK) can be used for adoptive cellular therapy, and different nano-drugs selected can play synergistic therapeutic effects with endogenous cells, so as to achieve the best therapeutic effect. Therefore, development of more safe and effective endogenous cell delivery systems is of great significance for improving the efficacy of nano-drugs or adoptive cellular therapy.

At present, in addition to a method of loading a nano-drug into a cell by means of the phagocytic function, the cell surface can also be modified with a nano-drug to construct a cell drug delivery system. Commonly used methods of loading a nano-drug on the cell surface are mainly as follows. (1) Chemical method: A nano-drug directly reacts with a functional group (such as a sulfydryl or amino group) on the cell surface. However, the cell surface does not necessarily contain sufficient free sulfydryl or amino groups, and the method of directly using the reactive groups on cell surface native proteins for carrying out a chemical reaction may affect normal physiological functions of cells. (2) Glycosylation method: An azide group (—N₃) is expressed on a cell membrane through glycosylation engineering, and then the cell surface is modified with a nano-drug by a chemical reaction. However, glycosylation engineering takes a long time and is not suitable for all cell types. (3) Genetic engineering method: Glycoprotein containing cyclooctyne is expressed on a cell surface by genetic engineering technology, and then the cell surface is modified with a nano-drug through a chemical reaction. This method requires specific biological technology for treating cells, and the treatment process is relatively complex, time-consuming and costly. (4) Physical method: This method is carried out through a receptor-ligand interaction or electrostatic interaction. However, this method is prone to endocytosis and is limited by overexpressed receptors on a cell surface, and the receptors occupied on the cell surface for a long time may also interfere with normal physiological functions of cells. Therefore, a new method for loading a nano-drug on a cell surface has broad application prospects and research value.

### SUMMARY

In view of the above-mentioned defects of the prior art, an objective of the present invention is to provide a method for anchoring and modifying a nano-drug on the surface of a cell.

Another objective of the present invention is to provide a living cell modified with a nano-drug prepared according to the method.

A third objective of the present invention is to provide an application of the living cell modified with a nano-drug.

The method for anchoring and modifying a nano-drug on the surface of a cell includes: introducing an active reactive group to the surface of a living cell by means of a hydrophobic tail chain of a cell membrane anchoring molecule; modifying the surface of a nano-drug with a corresponding reactive group; and carrying out a biological orthogonal click reaction between the active reactive group of the cell membrane anchoring molecule modified on the surface of the living cell and the corresponding reactive group modified on the surface of the nano-drug, so as to anchor and modify the nano-drug on the surface of the cell to obtain a living cell modified with the nano-drug.

The present invention discloses a cell membrane anchoring molecule, which can be anchored to the surface of a living cell and introduce an active reactive group ( [symbol] ) on the surface of the cell membrane. The structural general formula of the cell membrane anchoring molecule is as follows:

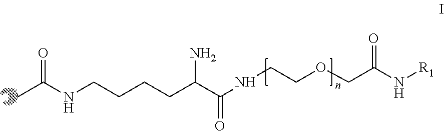

where R₁ is a common lipid or alkane chain, e.g. distearoyl phosphatidyl ethanolamine (DSPE), dioleoyl phosphatidyl ethanolamine (DOPE), 1,2-dihexadecyl-3-glycero-phosphoethanolamine (DHPE), cholesterol, long-chain alkanes with a chain length of 6-20 C, etc., preferably distearoyl phosphatidyl ethanolamine (DSPE);
n=8-200, preferably n=20-100.
[symbol] is an active reactive group, e.g. azide, azadibenzocyclooctyne, sulfydryl, amino, maleimide, α,β-unsaturated carbonyl, tetrazine, bicyclo[6.1.0]nonyne, etc., preferably tetrazine, bicyclo[6.1.0]nonyne, azide, and azadibenzocyclooctyne.

The present invention provides a method for synthesizing the above-mentioned cell membrane anchoring molecule, having a synthetic route as follows:
(1) Dissolving tetrazinic acid (hydrazoic acid, bicyclo[6.1.0]nonynoic acid, or azadibenzocyclooctynoic acid) and N-tert-butoxycarbonyl-L-lysine (Boc-Lys-OH) in chloroform (dichloromethane, or tetrahydrofuran); adding 1-ethyl-(3-dimethylaminopropyl) carbodiimide hydrochloride (EDCI) (or N,N-dicyclohexylcarbodiimide (DCC)), N-hydroxysuccinimide (NHS) and triethylamine (TEA) (or 4-dimethylaminopyridine (DMAP)); carrying out reaction at 25° C.-45° C. for 10-20 h; washing the reaction solution with water; drying the organic layer with anhydrous sodium sulfate (or anhydrous magnesium sulfate) and concentrating the organic layer; and carrying out column chromatography with dichloromethane/methanol to obtain a tetrazinated (azide, bicyclo[6.1.0]nonynylated, or azadibenzocyclooctynylated) derivative ( [symbol] Boc-Lys-OH). A synthetic reaction formula is as follows:

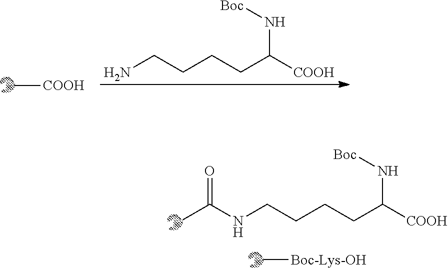

(2) Dissolving [symbol] Boc-Lys-OH and a PEG derivative with a molecular weight of 400-10000 in N,N-dimethylformamide (DMF) (or dimethyl sulfoxide (DMSO)); sequentially adding benzotriazol-1-yl-oxytripyrrolidinophosphonium hexafluorophosphate (PyBop) (1-ethyl-(3-dimethylaminopropyl) carbodiimide hydrochloride (EDC), or N-hydroxysuccinimide (NETS)), and triethylamine (TEA) (or N,N-diisopropylethylamine (DIPEA)); carrying out reaction at 25° C.-45° C. for 10-20 h; dialyzing the reaction solution for 24-48 h; and freeze-drying the reaction solution to obtain a tetrazine PEGylated (azide PEGylated, bicyclo[6.1.0]nonyne PEGylated, or azadibenzocyclooctyne PEGylated) derivative ( [symbol] Boc-Lys-PEG-R₁). A synthetic reaction formula is as follows:

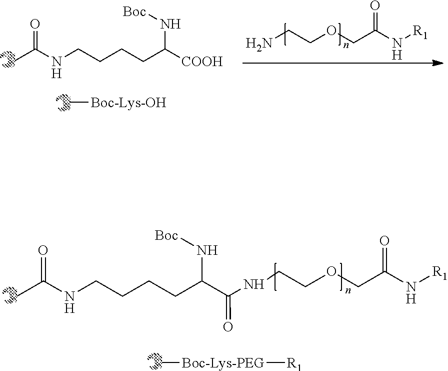

(3) Dissolving [symbol] Boc-Lys-PEG-R₁ in ethyl acetate/hydrochloric acid (dioxane/hydrochloric acid, or trifluoroacetic acid); carrying out reaction at 0° C.-45° C. for 2-20 h; dialyzing the reaction solution for 24-48 h; and freeze-drying the reaction solution to obtain a cell membrane anchoring molecule I. A synthetic reaction formula is as follows:

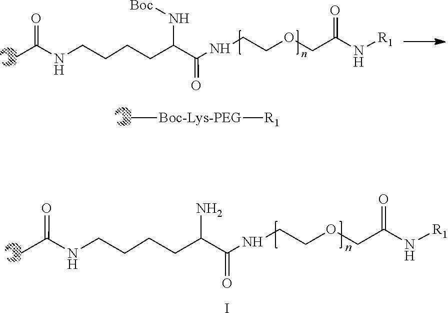

The living cells used in the present invention are preferably primary cells or immortalized cells with lipid membrane structures of humans or animals, including tumor cells, neutrophils, T cells, mesenchymal stem cells, hematopoietic stem cells, natural killer cells, antigen presenting cells, macrophages, etc., further preferably T cells or neutrophils. The T cells are selected from chimeric antigen receptor T cells, T cell receptor-gene engineered T cells or ordinary unmodified T cells.

The present invention discloses a nano-drug of which the surface is modified with a corresponding reactive group ( [symbol] ) the corresponding reactive group is introduced into the surface of a nanoparticle through a corresponding reactive group modifier, and the nano-drug is a nanoparticle loaded with a therapeutic agent.

The nanoparticles used in the present invention may be liposomes, nanovesicles, solid lipid nanoparticles, micelles, etc., preferably liposomes.

The therapeutic agent used in the present invention may be hydrophobic drugs such as avasimibe, paclitaxel, quercetin, BAY 87-2243, TGF-β inhibitors and piceatannol; hydrophilic drugs such as doxorubicin, daunorubicin and mitomycin; protein therapeutic drugs such as PD-1 monoclonal antibodies and PD-L1 monoclonal antibodies; and gene therapeutic drugs such as siRNA, mRNA, shRNA and plasmids, preferably avasimibe, paclitaxel, and PD-1 monoclonal antibodies.

The present invention further discloses a corresponding reactive group modifier, having a general structural formula as follows:

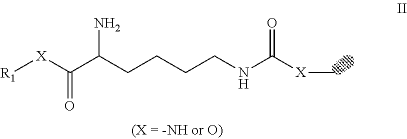

where R₁ is a common lipid or alkane chain, e.g. distearoyl phosphatidyl ethanolamine (DSPE), dioleoyl phosphatidyl ethanolamine (DOPE), 1,2-dihexadecyl-3-glycero-phosphoethanolamine (DHPE), cholesterol, long-chain alkanes with a chain length of 6-20 C, etc., preferably distearoyl phosphatidyl ethanolamine (DSPE).
[symbol] is a corresponding reactive group, e.g. azadibenzocyclooctyne, azide, maleimide, sulfydryl, amino, bicyclo[6.1.0]nonyne, tetrazine, etc., preferably bicyclo[6.1.0]nonyne, tetrazine, azadibenzocyclooctyne, and azide.

The present invention provides a method for synthesizing the above-mentioned corresponding reactive group modifier, having a synthetic route as follows:
(1) Dissolving hydroxylated (or aminated) bicyclo[6.1.0]nonyne (tetrazine, azadibenzocyclooctyne, or azide) with p-nitrophenyl chloroformate in dichloromethane (chloroform, or tetrahydrofuran); adding pyridine; carrying out reaction at 25° C.-40° C. for 4-10 h; concentrating the reaction solution; and carrying out column chromatography with dichloromethane/methanol to obtain p-nitrophenylated bicyclo[6.1.0]nonyne (tetrazine, azadibenzocyclooctyne, or azide), where a synthetic reaction formula is as follows:

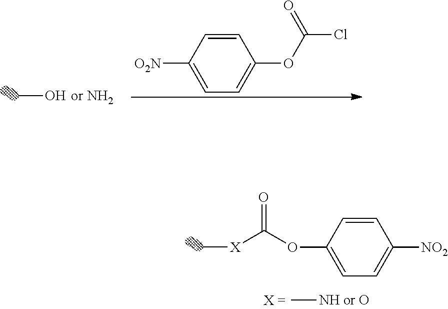

(2) Dissolving p-nitrophenylated bicyclo[6.1.0]nonyne (tetrazine, azadibenzocyclooctyne, or azide) and N-fluorenylmethoxycarbonyl-L-lysine (Fmoc-Lys-OH) in chloroform (dichloromethane, or tetrahydrofuran); adding 1-ethyl-(3-dimethylaminopropyl) carbodiimide hydrochloride (EDCI) (or N,N-dicyclohexylcarbodiimide (DCC)), N-hydroxysuccinimide (NHS) and triethylamine (TEA) (or 4-dimethylaminopyridine (DMAP)); carrying out reaction at 25° C.-45° C. for 10-20 h; washing the reaction solution with water; drying the organic layer with anhydrous sodium sulfate (or anhydrous magnesium sulfate) and concentrating the organic layer; and carrying out column chromatography with dichloromethane/methanol to obtain a bi cy clo[6.1. 0] nonynyl ated (tetrazinated, azadibenzocyclooctynylated, or azide) derivative ( [symbol] -Fmoc-Lys-OH). A synthetic reaction formula is as follows:

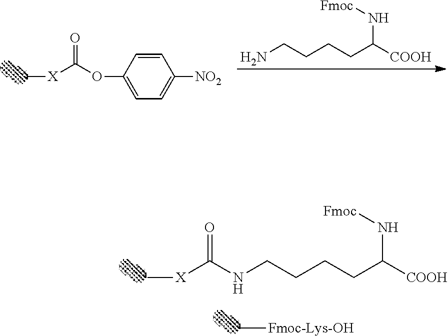

(3) Dissolving [symbol] -Fmoc-Lys-OH and an aminoated (or hydroxylated) phospholipid (cholesterol, or long-chain alkane) derivative in dichloromethane (chloroform, or tetrahydrofuran); adding 1-ethyl-(3 -dim ethyl aminopropyl) carb odiimi de hydrochloride (EDCI) (or N,N-dicyclohexylcarbodiimide (DCC)), N-hydroxysuccinimide (NHS) (or 1-hydroxybenzotriazole (HOB T)), and triethylamine (TEA) (or N,N-diisopropylethylamine (DIPEA)); carrying out reaction at 25° C.-45° C. for 3-24 h; washing the reaction solution with water; drying the organic layer with anhydrous sodium sulfate (or anhydrous magnesium sulfate) and concentrating the organic layer; and carrying out column chromatography with dichloromethane/methanol to obtain a bicyclo[6.1. 0] nonynyl ated (tetrazinated, azadibenzocyclooctynylated, or azide) phospholipid (cholesterol, or long-chain alkane) derivative ( [symbol] -Fmoc-Lys-R₁). A synthetic reaction formula is as follows:

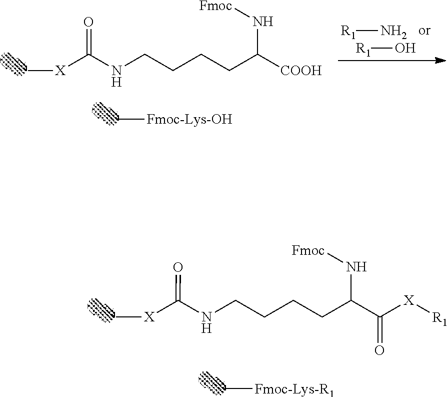

(4) Dissolving [symbol] -Fmoc-Lys-R₁ in chloroform (dichloromethane, or tetrahydrofuran); adding diethylamine (or piperidine); carrying out reaction at 0° C.-45° C. for 2-24 h; washing the reaction solution with water; drying the organic layer with anhydrous sodium sulfate (or anhydrous magnesium sulfate) and concentrating the organic layer; and carrying out column chromatography with dichloromethane/methanol to obtain a corresponding reactive group modifier II A synthetic reaction formula is as follows:

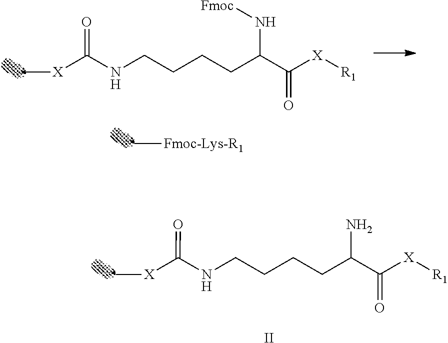

In the present invention, the particle size of the nano-drug is 1-1000 nm, preferably 10-500 nm; the drug loading of the therapeutic agent is 0.1%-20%, preferably 1%-15%; and the ratio of the corresponding reactive group modifier to the nanoparticles is 1:150-1:3, preferably 1:50-1:5.

The biological orthogonal click chemical reaction between the corresponding reactive group on the surface of the nano-drug and the active reactive group on the surface of the cell membrane according to the present invention includes ketone/hydroxylamine condensation, Michael addition of a sulfydryl or amino group with maleimide, strain-promoted azide-alkyne cycloaddition (SPAAC), and strain-promoted inverse electron-demand Diels-Alder cycloaddition (SPIEDAC), preferably SPAAC and SPIEDAC.

As a preference of the method of the present invention, the cell membrane anchoring molecule is incubated with a living cell at 0-40° C. for 5-120 min to obtain a living cell modified with the cell membrane anchoring molecule on the surface; and the nano-drug modified with the corresponding reactive group on the surface is incubated with the living cell modified with the cell membrane anchoring molecule on the surface at 0-37° C. for 5-120 min to obtain a living cell modified with the nano-drug.

In the incubation process of the cell membrane anchoring molecule and the living cell of the present invention, the concentration of the cell membrane anchoring molecule is preferably 10-200 μg/mL, the incubation time is preferably 10-60 min, and the incubation temperature is preferably 4-37° C.

In the incubation process of the nano-drug and the modified living cell according to the present invention, the concentration of the nano-drug is preferably 5-200 μg/mL, the incubation time is preferably 10-60 min, and the incubation temperature is preferably 4-37° C.

Based on the new technology for anchoring and modifying the surface of a living cell disclosed in the present invention, the present invention further discloses a living cell modified with a nano-drug, including a living cell, a cell membrane anchoring molecule and a nano-drug. First, the cell membrane anchoring molecule is incubated with the living cell for a period of time to prepare a living cell modified with an active reactive group. Then the nano-drug is incubated with the modified living cell, and the corresponding reactive group on the surface of the nano-drug and the active reactive group on the surface of the cell membrane undergo a biological orthogonal click reaction, so as to stably anchor the nano-drug to the surface of the living cell to form a cell drug (FIG. 1). The cell drug can prolong the in vivo circulation time of the nano-drug by means of the physiological/pathological properties of the living cell, improve the targeting efficiency of the nano-drug to a specific site, and also induce a synergistic therapeutic effect between the nano-drug and the living cell. In consequence, depending on the type of living cells and therapeutic agents used, cell drugs are used for treating various diseases.

As a preference of the present invention, the present invention claims to protect a T cell modified with a nano-drug prepared according to the method, further preferably a chimeric antigen receptor T cell or a T cell receptor-gene engineered T cell modified with a nano-drug prepared according to the method of the present invention.

The cell drug of the present invention has a survival rate of greater than 80% of the living cell, and a drug loading of 0.1-20 μg/10⁶ cells, and maintains normal physiological function of the living cell, including cell proliferation, cell chemotaxis, cell activation, etc.

The present invention provides an application of the living cell modified with a nano-drug of the present invention in preparation of a drug for treating tumors or inflammation-related diseases.

The tumors include melanoma, glioma, breast cancer or ovarian cancer. The inflammation-related diseases include stroke or arthritis.

The present invention provides an application of the cell membrane anchoring molecule of the present invention in preparation of a living cell drug, where the living cell drug is a living cell modified with a nano-drug on the surface.

The present invention provides an application of the corresponding reactive group modifier of the present invention in preparation of a living cell drug, where the living cell drug is a living cell modified with a nano-drug on the surface, preferably a T cell modified with a nano-drug on the surface, further preferably a chimeric antigen receptor T cell or a T cell receptor-gene engineered T cell modified with a nano-drug on the surface.

### Beneficial Effects:

The present invention develops a new method for loading a nano-drug on the surface of a cell. The method simulates a phospholipid hydrophobic tail chain of a (glycosyl phosphatidyl inositol) GPI anchor to introduce a chemical reactive group to the surface of the cell membrane, and then the surface of the cell is chemically modified with a nano-drug modified with a corresponding reactive group on the surface, so as to obtain the corresponding cell drug for treating various diseases. The new loading method introduces the reactive group into the surface of the cell through hydrophobic interaction, does not interfere with the gene, metabolism and native protein activity of the cell, has relatively little effects on the cell, and is suitable for any cell with a lipid membrane structure. In summary, the new cell loading technology studied herein is safe, stable, efficient, and broad-spectrum, has unique advantages compared with other methods, and can be used for treating various diseases according to the nano-drugs loaded and the cell types used.

The cell surface anchoring technology disclosed in the present invention is simple, convenient, quick and in common use, and can be applied to various cells with lipid membrane structures, including primary cells, e.g. human T cells (Embodiments 12 and 13), human CAR-T cells (Embodiments 14 and 15), murine T cells (Embodiment 16), murine TCR-T cells (Embodiment 17), human neutrophils (Embodiments 18 and 19), murine neutrophils (Embodiment 20), mesenchymal stem cells (Embodiment 21), and tumor cells, such as lung cancer cells A549 (Embodiment 22). The function of the cells themselves are not be affected (Embodiments 25-27) after such a modification. The method provides a new technical platform for cell modification and has very wide application prospects.

Compared with pure cells and pure nano-drugs, a cell drug obtained by the above-mentioned cell modification technology disclosed by the present invention has an optimal treatment effect (Embodiments 28-30) and provides a new idea and a new drug for treating various diseases.

### BRIEF DESCRIPTION OF THE DRAWINGS

FIG. 1 shows a preparation flow chart of a cell drug of the present invention.

FIG. 2 shows an ultraviolet spectrum of a cell membrane anchoring molecule of the present invention reacted with a corresponding reactive group modifier.

FIG. 3 is a transmission electron micrograph of a nano-drug of the present invention.

FIG. 4 is a laser confocal image of a cell drug of the present invention.

FIG. 5 shows detection of the viability of a cell drug of the present invention.

FIG. 6 shows characterization of the proliferation ability of a cell drug of the present invention.

FIG. 7 shows characterization of the chemotactic ability of a cell drug of the present invention.

FIG. 8 shows a tumor inhibition curve of a cell drug of the present invention in treating melanoma in situ and a tumor tissue picture.

FIG. 9 shows a tumor inhibition curve of a cell drug of the present invention in treating breast cancer in situ.

FIG. 10 is a picture showing the effect of a cell drug of the present invention in treating glioma in situ.

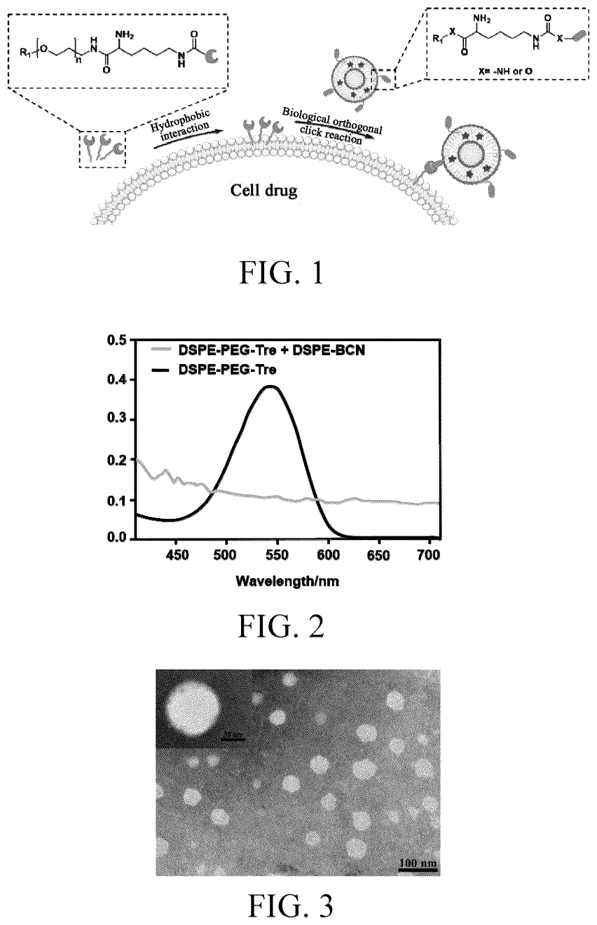

### DETAILED DESCRIPTION

### EMBODIMENT 1

Preparation and characterization of cell membrane anchoring molecule--distearoyl phosphatidyl ethanolamine-polyethylene glycol 5000-lysine-tetrazine (DSPE-PEG₅ₖ-Tre)

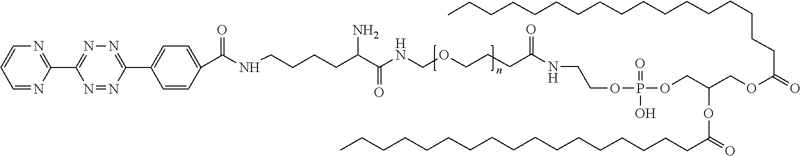

4-(6-(pyrimidin-2-yl)-1,2,4,5-tetrazin-3-yl) benzoic acid (tetrazinic acid (Tre-COOH), 80 mg, 0.29 mmol) and N-tert-butoxycarbonyl lysine hydrochloride (Boc-Lys-OH.HC1, 126.42 mg, 0.26 mmol) were dissolved in chloroform (30 mL), and N-hydroxysuccinimide (NHS, 35.68 mg, 0.31 mmol), 1-(3-dimethylaminopropyl)-3-ethylcarbodiimide hydrochloride (EDCI, 59.43 mg, 0.31 mmol), and DIPEA (136.24 μL, 100.82 mg, 0.78 mmol) were added to react overnight at room temperature. The reaction solution was washed with water and dried with anhydrous sodium sulfate. The organic layer was concentrated and subjected to column chromatography with dichloromethane/methanol to obtain a purplish red powdery solid (N²-(tert-butoxycarb onyl)-N⁶-(4-(6-(pyrimidin-2-yl)-1,2,4,5-tetrazin-3 -yl)benzoyl)lysine, 90 mg, 61.9%). Distearoyl phosphatidyl ethanolamine-polyethylene glycol 5000-amino (50 mg, 0.01 mmol) was dissolved in DMF (5 mL), and benzotriazol-1-yl-oxytripyrrolidinophosphonium hexafluorophosphate (PyBop, 11.45 mg, 0.022 mmol), triethylamine (4.09 μL, 3.03 mg, 0.03 mmol) and (N²-(tert-butoxy c arb onyl)-N⁶-(4-(6-(pyrimi din-2-yl)-1,2,4,5-tetrazin-3 -yl)benzoyl)lysine (10.52 mg, 0.02 mmol) were added sequentially and stirred overnight. The reaction solution was placed in a dialysis bag, dialyzed with dimethyl sulfoxide as a dialysis medium for 48 h, continued to be dialyzed with deionized water for 48 h, and freeze-dried to obtain a purplish red flocculent product (distearoyl phosphatidyl ethanolamine-polyethylene glycol 5000-N²-(tert-butoxycarb onyl)-N⁶-(4-(6-(pyrimidin-2-yl)-1,2,4,5-tetrazin-3-yl)benzoyl)lysine, 31.7 mg, 60.8%). The distearoyl phosphatidyl ethanolamine-polyethylene glycol 5000-N 2 -(tert-butoxycarb onyl)-/V 6 -(4-(6-(pyrimidin-2-yl)-1,2,4,5-tetrazin-3-yl)benzoyl)lysine (31.7 mg) was dissolved in deionized water (5 mL), and trifluoroacetic acid (TFA, 50 μL) was added and stirred overnight. The reaction solution was then transferred to a dialysis bag, dialyzed with deionized water as a dialysis medium for 48 h, and freeze-dried to obtain a purplish red flocculent product (distearoyl phosphatidyl ethanolamine-polyethylene glycol 5000-lysine-tetrazine, 20 mg).

¹-NMR (300 MHz, d⁶-DMSO): δ 9.21 (2H, d), 8.68 (1H, d), 8.19 (2H, d), 7.51 (2H, d), 5.11-5.19 (4H, m), 4.57-4.52 (7H, m), 4.10-3.99 (9H, m), 3.77-3.68 (8H, m), 3.53-3.46 (475H, m), 2.32-2.19 (5H, m), 1.56-1.40 (7H, m), 1.25-1.20 (45H, m), 0.85 (6H, t).

### EMBODIMENT 2

Preparation and characterization of cell membrane anchoring molecule--dioleoyl phosphatidyl ethanolamine-polyethylene glycol 2000-lysine-sulfydryl (DOPE-PEG₂ₖ-SH)

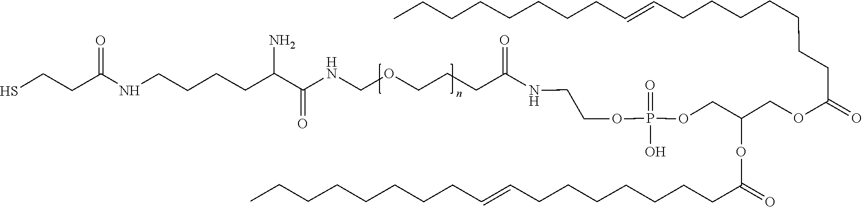

Mercaptopropionic acid (SH—COOH, 30 mg, 0.29 mmol) and N-tert-butoxycarbonyl lysine hydrochloride (Boc-Lys-OH.HC1, 126.42 mg, 0.26 mmol) were dissolved in chloroform (30 mL), and N-hydroxysuccinimide (NHS, 35.68 mg, 0.31 mmol), 1-(3-dimethylaminopropyl)-3-ethylcarbodiimide hydrochloride (EDCI, 59.43 mg, 0.31 mmol), and DIPEA (136.24 μL, 100.82 mg, 0.78 mmol) were added to react at room temperature overnight. The reaction solution was washed with water and dried with anhydrous sodium sulfate. The organic layer was concentrated and subjected to column chromatography with dichloromethane/methanol to obtain a faint yellow solid (N²-(tert-butoxycarbonyl)-N⁶-(3-mercaptopropionyl)lysine, 82 mg, 85.4%). Dioleoyl phosphatidyl ethanolamine-polyethylene glycol 2000-amino (20 mg) was dissolved in DMF (5 mL), and benzotriazol-1-yl-oxytripyrrolidinophosphonium hexafluorophosphate (PyBop, 11.45 mg, 0.022 mmol), triethylamine (4.09 μL, 3.03 mg, 0.03 mmol) and (N²-(tert-butoxycarbonyl)-N⁶-(3-mercaptopropionyl)lysine, 6.68 mg, 0.02 mmol) were added sequentially and stirred overnight. The reaction solution was placed in a dialysis bag, dialyzed with dimethyl sulfoxide as a dialysis medium for 48 h, continued to be dialyzed with deionized water for 48 h, and freeze-dried to obtain a faint yellow flocculent product (dioleoyl phosphatidyl ethanolamine-polyethylene glycol 2000-N²-(tert-butoxycarbonyl)-N⁶-(3-mercaptopropionyl)lysine, 21.7 mg, 54.2%). The dioleoyl phosphatidyl ethanolamine-polyethylene glycol 2000-N²-(tert-butoxycarbonyl)-N⁶-(3-mercaptopropionyl)lysine (21.7 mg) was dissolved in deionized water (5 mL), and trifluoroacetic acid (TFA, 50 μL) was added and stirred overnight. The reaction solution was then transferred to a dialysis bag, dialyzed with deionized water as a dialysis medium for 48 h, and freeze-dried to obtain a faint yellow flocculent product (dioleoyl phosphatidyl ethanolamine-polyethylene glycol 2000-lysine-sulfydryl, 10 mg).

¹-NMR (300 MHz, d⁶-DMSO): δ 5.26 (4H, m), 5.11-5.19 (4H, m), 4.57-4.52 (9H, m), 4.10-3.99 (9H, m), 3.62-3.56 (8H, m), 3.53-3.46 (184H, m), 2.52-2.29 (7H, m), 1.59-1.43 (7H, m), 1.25-1.20 (45H, m), 0.85 (6H, t).

### EMBODIMENT 3

Preparation and characterization of cell membrane anchoring molecule--stearyl alcohol-glutamate-polyethylene glycol 1000-lysine-azide (SA₂-Glu-PEG₁ₖ-N₃)

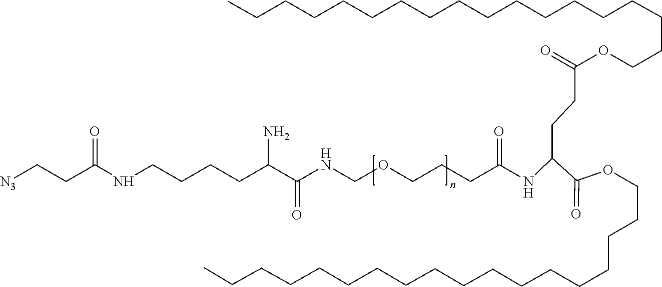

Azidopropionic acid (N₃—COOH, 33 mg, 0.29 mmol) and N-tert-butoxycarbonyl lysine hydrochloride (Boc-Lys-OH·HCl, 126.42 mg, 0.26 mmol) were dissolved in chloroform (30 mL), and N-hydroxysuccinimide (NHS, 35.68 mg, 0.31 mmol), 1-(3-dimethylaminopropyl)-3-ethylcarbodiimide hydrochloride (EDCI, 59.43 mg, 0.31 mmol), and DIPEA (136.24 μL, 100.82 mg, 0.78 mmol) were added to react at room temperature overnight. The reaction solution was washed with water and dried with anhydrous sodium sulfate. The organic layer was concentrated and subjected to column chromatography with dichloromethane/methanol to obtain a white solid (N²-(tert-butoxycarbonyl)-N⁶-(3-azidopropionyl)lysine, 90 mg, 90.4%). Stearyl alcohol-glutamate-polyethylene glycol 1000-amino (20 mg) was dissolved in DMF (5 mL), and benzotriazol-1-yl-oxytripyrrolidinophosphonium hexafluorophosphate (PyBop, 11.45 mg, 0.022 mmol), triethylamine (4.09 pL, 3.03 mg, 0.03 mmol) and (N²-(tert-butoxycarbonyl)-N⁶-(3-azidopropionyl)lysine, 6.86 mg, 0.02 mmol) were added sequentially and stirred overnight. The reaction solution was placed in a dialysis bag, dialyzed with dimethyl sulfoxide as a dialysis medium for 48 h, continued to be dialyzed with deionized water for 48 h, and freeze-dried to obtain a white flocculent product (stearyl alcohol-glutamate-polyethylene glycol 1000-N²-(tert-butoxycarbonyl)-N⁶-(3-azidopropionyl)lysine, 21.7 mg, 40.5%). The stearyl alcohol-glutamate-polyethylene glycol 1000-N²-(tert-butoxycarbonyl)-N⁶-(3-azidopropionyl)lysine (21.7 mg) was dissolved in deionized water (5 mL), and trifluoroacetic acid (TFA, 50 μL) was added and stirred overnight. The reaction solution was then transferred to a dialysis bag, dialyzed with deionized water as a dialysis medium for 48 h, and freeze-dried to obtain a faint yellow flocculent product (stearyl alcohol-glutamate-polyethylene glycol 1000-lysine-azide, 10 mg). ¹H-NMR (300 MHz, d⁶-DMSO): δ 5.37 (4H, m), 5.16-5.09 (4H, m), 4.38-4.22 (9H, m), 4.10-3.99 (9H, m), 3.62-3.56 (8H, m), 3.53-3.46 (83H, m), 2.62-2.33 (7H, m), 1.59-1.43 (7H, m), 1.27-1.22 (69H, m), 0.85 (6H, t).

### EMBODIMENT 4

Preparation and characterization of corresponding reactive group modifier--distearoyl phosphatidyl ethanolamine-lysine-cyclononyne (DSPE-BCN)

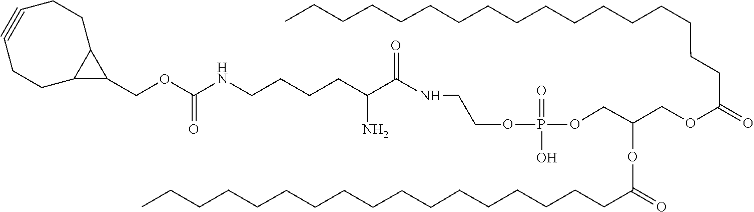 [SMILES: CCCCCCCCCCCCCCCCCC(=O)OCC(COP(=O)(O)OCCNC(=O)C(N)CCCCNC(=O)OCC1C2CCC#CCCC21)OC(=O)CCCCCCCCCCCCCCCCC]

Bicyclo[6.1.0]non-4-yn-9-ylmethanol (350 mg, 2.33 mmol) was dissolved in dichloromethane (30 mL), and p-nitrophenyl chloroformate (1.17 g, 5.82 mmol) and pyridine (Py, 0.64 g, 8.15 mmol) were added to react at room temperature for 6 h. The reaction solution was concentrated and subjected to column chromatography to obtain a white powdery solid (bicyclo[6.1.0]non-4-yn-9-ylmethyl-(4-nitrophenyl)carbamate, 520 mg, 71.1%). The bicyclo[6.1.0]non-4-yn-9-ylmethyl-(4-nitrophenyl)carbamate (360 mg, 1.14 mmol) was dissolved in 5 mL DMF, and N-fluorenylmethoxycarbonyl-L-lysine (612 mg, 1.26 mmol), and DIPEA (0.65 mL, 3.77 mmol) were added sequentially to react for 4 h. The reaction solution was washed with a sodium citrate aqueous solution and a saturated salt solution, dried with anhydrous sodium sulfate, concentrated and purified by column chromatography to obtain a white oily solid (N²-(((9H-fluoren-9-yl)methoxy)carb onyl)-N⁶-((bicyclo [6. 1. O]non-4-yn-9-ylmethoxy)carb on yl)lysine, 320 mg, 51.6%). The N²-(9H-fluoren-9-yl)methoxy)carbonyl)-N⁶-((bicyclo[6.1.0]non-4-yn-9-ylmethoxy)carbonyl)lysine(100 mg, 0.18 mmol), N-hydroxysuccinimide (NHS, 26 mg, 0.12 mmol), 1-(3-dimethylaminopropyl)-3-ethylcarbodiimide hydrochloride (EDCI, 45 mg, 0.12 mmol), and distearoyl phosphatidyl ethanolamine (DSPE, 137 mg, 0.202 mmol) were dissolved in chloroform (20 mL), and DIPEA (106 μL, 0.30 mmol) was added to react at room temperature overnight. The reaction solution was washed with an aqueous citric acid solution (2×80 mL) and a saturated salt solution (2×80 mL). The organic phase was collected, dried with anhydrous sodium sulfate, concentrated by distillation under reduced pressure, and purified by column chromatography to obtain a light pink powdery solid (1-(((2-(2-((((9H-fluoren-9-yl)methoxy)carb onyl)amino)-6-(((bicyclo[6. 1. O]non-4-yn-9-ylmet hoxy)carbonyl)amino)hexamido)ethoxy)(hydroxy)phosphoryl)oxy)ethane-1,2-diyldistearate, 200 mg, 88.5%). 10 mL dichloromethane was added to a 50 mL eggplant-shaped flask, and then the 1(((2-(2-((((9H-fluoren-9-yl)methoxy)carb onyl)amino)-6-(((bicyclo[6. 1. O]non-4-yn-9-ylmet hoxy)carbonyl)amino)hexamido)ethoxy)(hydroxy)phosphoryl)oxy)ethane-1,2-diyldistearate (100 mg) was added. After fully dissolving, diethylamine was added to react overnight. The reaction solution was fully concentrated and purified by column chromatography to obtain a white powdery solid (distearoyl phosphatidyl ethanolamine-lysine-cyclononyne, 50 mg, 61.3%) finally.

MS, ESI⁻, m/z: calcd for C₅₈H₁₀₆N₃O₁₁P (M-H)⁻ 1050.8 found 1050.8, (M+H₂O—H)⁻ 1068.8 found 1068.8. ¹-NMR (300 MHz, CDC1 3): δ 5.42 (1H, m), 5.11 (1H, m), 4.40-4.26 (1H, m), 4.09-4.03 (1H, m), 3.90-3.77 (6H, m), 3.67-3.54 (2H, m), 3.07 (2H, m), 2.32-2.09 (8H, m), 1.78 (4H, m), 1.50-1.28 (8H, m), 1.28-1.17 (58H, m), 0.80 (6H, t), 0.61-0.55 (3H, m).

### EMBODIMENT 5

Preparation and characterization of corresponding reactive group modifier--tetradecyl alcohol-glutamate-lysine-maleimide (TA₂-Glu-Lys-Mal)

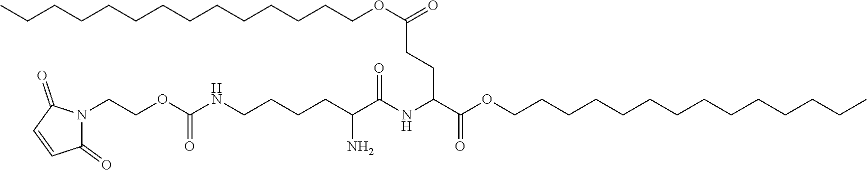 [SMILES: CCCCCCCCCCCCCCOC(=O)CCC(NC(=O)C(N)CCCCNC(=O)OCCN1C(=O)C=CC1=O)C(=O)OCCCCCCCCCCCCCC]

N-hydroxyethylmaleimide (Mal-OH, 328 mg, 2.33 mmol) was dissolved in dichloromethane (30 mL), and p-nitrophenyl chloroformate (1.17 g, 5.82 mmol) and pyridine (Py, 0.64 g, 8.15 mmol) were added to react at room temperature for 6 h. The reaction solution was concentrated and subjected to column chromatography to obtain a solid (2-maleimide-(4-nitrophenyl)carbamate, 520 mg, 73.2%). The 2-maleimide-(4-nitrophenyl)carbamate (347 mg, 1.14 mmol) was dissolved in 5 mL DMF, and N-fluorenylmethoxycarbonyl-L-lysine (612 mg, 1.26 mmol), and DIPEA (0.65 mL, 3.77 mmol) were added sequentially to react for 4 h. The reaction solution was washed with a sodium citrate aqueous solution and a saturated salt solution, dried with anhydrous sodium sulfate, concentrated and purified by column chromatography to obtain a white oily solid (N²-(((9H-fluoren-9-yl)methoxy)carbonyl)-N⁶-((2-maleimide)carbamate)lysine, 320 mg, 68%). The N²-(((9H-fluoren-9-yl)methoxy)carbonyl)-N⁶-maleimide)carbamate)lysine (74.34 mg, 0.18 mmol), N-hydroxysuccinimide (NHS, 26 mg, 0.12 mmol), 1-(3-dimethylaminopropyl)-3-ethylcarbodiimide hydrochloride (EDCI, 45 mg, 0.12 mmol), and tetradecyl alcohol-glutamate (TA₂-Glu, 109 mg, 0.202 mmol) were dissolved in chloroform (20 mL), and DIPEA (106 μL, 0.30 mmol) was added to react at room temperature overnight. The reaction solution was washed with an aqueous citric acid solution (2×80 mL) and a saturated salt solution (2×80 mL). The organic phase was collected, dried with anhydrous sodium sulfate, concentrated by distillation under reduced pressure, and purified by column chromatography to obtain a solid (tetradecyl alcohol-glutamate-N²-(((9H-fluoren-9-yl)methoxy)carbonyl)-N⁶-((2-maleimide)carbamate)lys ine, 150 mg, 89%). 10 mL dichloromethane was added to a 50 mL eggplant-shaped flask, and then the tetradecyl alcohol-glutamate-N²-(((9H-fluoren-9-yl)methoxy)carbonyl)-N⁶-((2-maleimide)carbamate)lys ine (93.4 mg, 0.1 mmol) was added. After fully dissolving, diethylamine was added to react overnight. The reaction solution was fully concentrated and purified by column chromatography to obtain a white solid (tetradecyl alcohol-glutamate-lysine-maleimide, 57 mg, 68%) finally.

MS, ESI-, m/z: calcd for C₄₂H₈₂N₄O₆S (M+H)⁺ 835.6115 found 835.6024. ¹-NMR (300 MHz, CDCl₃): δ 7.86 (2H, s), 4.55 (1H, m), 4.20-4.06 (4H, m), 3.46 (2H, t), 3.25 (1H, m), 3.04 (2H, m), 2.82-2.39 (6H, q), 1.80-1.75 (2H, m), 1.62-1.17 (52H, m), 0.88 (6H, t).

### EMBODIMENT 6

Synthesis and characterization of corresponding reactive group modifier--cholesterol-lysine-cyclooctyne (Chol-Lys-ADIBO)

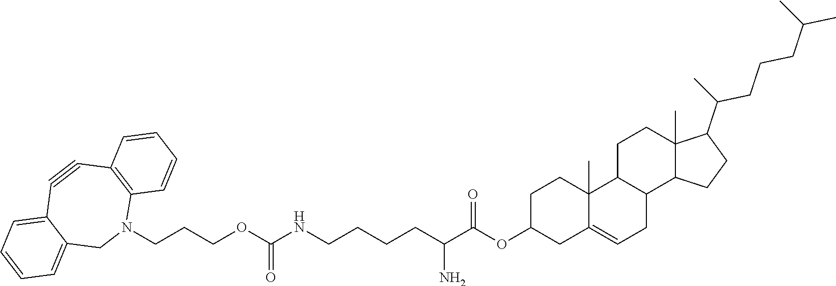 [SMILES: CC(C)CCCC(C)C1CCC2C3CC=C4CC(OC(=O)C(N)CCCCNC(=O)OCCCN5Cc6ccccc6C#Cc6ccccc65)CCC4(C)C3CCC12C]

N-((3-hydroxy)-5,6-dihydrodibenzo[b,f]azeticyclooctyne (hydroxylated azadibenzocyclooctyne, 643 mg, 2.33 mmol) was dissolved in dichloromethane (30 mL), and p-nitrophenyl chloroformate (1.17 g, 5.82 mmol) and pyridine (Py, 0.64 g, 8.15 mmol) were added to react at room temperature for 6 h. The reaction solution was concentrated and subjected to column chromatography to obtain a white solid (1-(N-((3 -hy droxy)-5, 6-dihydrodibenzo[b,f]azeticyclooctyne)-(4-nitrophenyl)carbamate, 830 mg, 80.7%). The 1-(N-((3-hydroxy)-5,6-dihydrodibenzo[b,f]azeticyclooctyne)(4-nitrophenyl)carbamate (500 mg, 1.14 mmol) was dissolved in 5 mL DMF, and N-fluorenylmethoxycarbonyl-L-lysine (612 mg, 1.26 mmol), and DIPEA (0.65 mL, 3.77 mmol) were added sequentially to react for 4 h. The reaction solution was washed with a sodium citrate aqueous solution and a saturated salt solution, dried with anhydrous sodium sulfate, concentrated and purified by column chromatography to obtain a white oily solid (N²-(((9H-fluoren-9-yl)methoxy)carb onyl)-N⁶-(N-((3-hydroxy)-5, 6-di hy drodib enzo [b, f]azetic yclooctyne)carbamate)lysine, 520 mg, 68%). The N²(((9H-fluoren-9-yl)methoxy)carbonyl)-N⁶-(N-((3-hydroxy)-5, 6-di hy drodib enzo [b,f]azetic yclooctyne)carbamate)lysine (120 mg, 0.18 mmol), N-hydroxysuccinimide (NHS, 26 mg, 0.12 mmol), 1-(3-dimethylaminopropyl)-3-ethylcarbodiimide hydrochloride (EDCI, 45 mg, 0.12 mmol), and cholesterol (Chol, 78 mg, 0.202 mmol) were dissolved in chloroform (20 mL), and DIPEA (106 μL, 0.30 mmol) was added to react at room temperature overnight. The reaction solution was washed with an aqueous citric acid solution (2×80 mL) and a saturated salt solution (2×80 mL). The organic phase was collected, dried with anhydrous sodium sulfate, concentrated by distillation under reduced pressure, and purified by column chromatography to obtain a solid (cholesterol-N²-(((9H-fluoren-9-yl)methoxy)carbonyl)-N⁶-(N-((3 -hy droxy)-5, 6-dihydrodiben zo[b,f]azeticyclooctyne)carbamate)lysine, 150 mg, 80.6%). 10 mL dichloromethane was added to a 50 mL eggplant-shaped flask, and then the cholesterol-N²(((9H-fluoren-9-yl)methoxy)carbonyl)-N⁶-(N-((3 -hy droxy)-5, 6-dihydrodibenz o[b,f]azeticyclooctyne)carbamate)lysine (103 mg, 0.1 mmol) was added. After fully dissolving, diethylamine was added to react overnight. The reaction solution was fully concentrated and purified by column chromatography to obtain a white solid (cholesterol-lysine-cyclooctyne, 56 mg, 68.5%) finally.

MS, ESI-, m/z: calcd for C₅₂H₇₃N₃O₄ (M+H)⁺ 804.5634 found 804.5665. ¹H-NMR (300 MHz, CDCl₃): δ 7.63 (1H, d), 7.32 (5H, m), 7.21 (2H, m), 5.37 (1H, m), 5.18 (2H, d), 4.63 (1H, m), 3.41 (1H, m), 3.22 (2H, t), 3.10 (2H, t), 2.66 (2H, m), 2.31 (2H, m), 1.99 (4H, m), 1.84 (5H, m), 1.53 (8H, m), 1.31 (8H, m), 1.12 (8H, m), 1.02 (6H, m), 0.92 (4H, m), 0.86 (6H, m), 0.68 (3H, m).

### EMBODIMENT 7

Biological orthogonal click reaction between cell membrane anchoring molecule and corresponding reactive group modifier

Taking DSPE-PEG₅ₖ-Tre and DSPE-BCN as examples, the tetrazine group (Tre) has an obvious characteristic absorption peak around 540 nm. The UV absorption peak at 540 nm disappeared when the tetrazine group (Tre) underwent an SPIEDAC reaction with bicyclo[6.1.0]nonyne (BCN). The cell membrane anchoring molecule (DSPE-PEG 5k -Tre) was dissolved in chloroform, and then a chloroform solution of the corresponding reactive group modifier (DSPE-BCN) was added to react at room temperature. Wavelength scanning was carried out on the reaction solution by a UV spectrophotometer, and also on the chloroform solution of the DSPE-PEG₅ₖ-Tre, and an absorption curve was drawn. The results are shown in FIG. 2. From FIG. 2, the characteristic absorption peak of tetrazine in the reaction solution of the DSPE-PEG₅ₖ-Tre and the DSPE-BCN disappeared at about 540 nm, indicating that the biological orthogonal click reaction between the two is basically complete. Therefore, a mild and efficient click chemical reaction can take place between the cell membrane anchoring molecule and the corresponding reactive group modifier.

### EMBODIMENT 8

Preparation and characterization of liposome nano-drug (BCN-Ava-Lip) modified with corresponding reactive group

100 mg of commercially available soybean phospholipid (SPC), 15 mg of cholesterol, and 3 mg of avasimibe (Ava) were added to 25 mg of the corresponding reactive group modifier (DSPE-BCN), and dissolved in chloroform and methanol. The organic solvent was removed by rotary evaporation for 5 min. The reaction solution was dried in vacuum overnight, hydrated at 37° C. for 30 min, sonicated with a probe for 10-30 min, and then passed through 0.80, 0.45, and 0.22 μm filter membranes in turn to obtain a DSPE-BCN-modified liposome (BCN-Ava-Lip). The nano-drug (BCN-Ava-Lip) modified with the corresponding reactive group measured has a particle size of 91.5±1.4 nm, a drug loading of 2.3%, and an encapsulation efficiency of 89.1%.

### EMBODIMENT 9

Preparation and characterization of liposome nano-drug (Mal-siRNA-Lip) modified with corresponding reactive group

15 mg of SPC, 8 mg of the corresponding reactive group modifier (TA₂-Glu-Lys-Mal), 15 mg of cationic lipid material, and 9 mg of cholesterol were dissolved in chloroform and methanol. The organic solvent was removed by rotary evaporation. The reaction solution was dried in vacuum overnight, hydrated at 37° C. for 30 min, sonicated with a probe for 10-30 min, and then passed through 0.80, 0.45, and 0.22 1.tm filter membranes in turn to obtain a TA₂-Glu-Lys-Mal-modified blank liposome (Mal-Lip). 14 μL of Mal-Lip (9.4 mg/mL) was diluted by adding 186 μL of ultrapure water, 10 μL of siRNA (0.5 mg/mL) was diluted by adding 190 μL of ultrapure water, and the two dilutions were mixed by vortex (N/P=5), and incubated at room temperature for 30 min to obtain a liposome (Mal-siRNA-Lip) modified with TA₂-Glu-Lys-Mal and loaded with siRNA. The nano-drug (Mal-siRNA-Lip) modified with the corresponding reactive group measured has a particle size of 117.3±2.8 nm, and an encapsulation efficiency of 100%.

### EMBODIMENT 10

Preparation and characterization of solid lipid nanoparticle drug modified with corresponding reactive group (ADIBO-PTX-NPs)

3 mg of poloxamer was dissolved in ultrapure water and heated to 75° C. to be used as a water phase. 3 mg of paclitaxel (PTX), 30 mg of glycerol monostearate, and 15 mg of the corresponding reactive group modifier (Chol-Lys-ADIBO) were accurately weighed, a small amount of ethanol was added, and the mixed solution was stirred and melted at 75° C. to be used as an oil phase. When the two phases were completely dissolved and were at the same temperature, the water phase was poured into the oil phase and stirred quickly to mix thoroughly. The mixed solution was volatilized until there was no alcohol smell, sonicated for 5 min, and cooled at room temperature to obtain a Chol-Lys-ADIBO-modified solid lipid nanoparticle (ADIBO-PTX-NPs). The nano-drug (ADIBO-PTX-NPs) modified with the corresponding reactive group measured has a particle size of 165.3±1.1 nm, a drug loading of 5.6%, and an encapsulation efficiency of 90%.

### EMBODIMENT 11

Electron microscopy characterization of nano-drug

Taking BCN-Ava-Lip as an example, a nano-drug solution was diluted to a certain concentration, added dropwise to a copper mesh covered with a carbon film, and allowed to stand at room temperature. The excess solution was absorbed with filter paper, the nano-drug was negatively stained with a 0.1% sodium phosphotungstate solution, and the stain was washed off After drying at room temperature, the nano-drug was observed and a picture was taken with a HT-7700 transmission electron microscope (100 kV). The TEM image is shown in FIG. 3. The result shows that the nano-drug BCN-Ava-Lip is nearly spherical in shape and uniform in particle size.

### EMBODIMENT 12

Preparation of human T cell drug (BCN-Ava-Lip/hT cell)

The density of a human peripheral blood-derived T cell (hT cell) suspension was adjusted to 1×10⁶ cells/mL. A certain amount of cell membrane anchoring molecules (DSPE-PEG₅ₖ-Tre) were added to each milliliter of cell suspension, and the cell suspension was incubated at 4° C. for 30 min, and centrifuged at 1500 rmp for 5 min. The supernatant was discarded, and cells were washed 2-3 times with PBS and resuspended to obtain hT cells with reactive groups on the surface. The nano-drug BCN-Ava-Lip was adjusted to isotonic and diluted into a solution with an avasimibe concentration of 150 μg/mL. The solution was incubated with the hT cells with active reactive groups on the surface at 25° C. for 20 min, and centrifuged at 1500 rmp for 5 min. The supernatant was discarded, and cells were washed with PBS to remove the unreacted nano-drug, and resuspended to obtain human T cells modified with the nano-drug on the surface, namely a BCN-Ava-Lip/hT cell drug.

### EMBODIMENT 13

Preparation of human T cell drug (ADIBO-PTX-NPs/hT cell)

The density of a human peripheral blood-derived T cell (hT cell) suspension was adjusted to 1×10⁶ cells/mL. A certain amount of cell membrane anchoring molecules (SA₂-Glu-PEG₁ₖ-N₃) were added to each milliliter of cell suspension, and the cell suspension was incubated at 4° C. for 20 min, and centrifuged at 1500 rmp for 5 min. The supernatant was discarded, and cells were washed 2-3 times with PBS and resuspended to obtain hT cells with reactive groups on the surface. The nano-drug (ADIBO-PTX-NPs) was adjusted to isotonic and diluted into a solution with a paclitaxel concentration of 100 μg/mL. The solution was incubated with the hT cells with active reactive groups on the surface at 37° C. for 45 min, and centrifuged at 1500 rmp for 5 min. The supernatant was discarded, and cells were washed with PBS to remove the unreacted nano-drug, and resuspended to obtain human T cells modified with the nano-drug on the surface, namely an ADIBO-PTX-NPs/hT cell drug.

### EMBODIMENT 14

Preparation of CAR-T cell drug (BCN-Ava-Lip/CAR-T cell)

1 mL of 10 μg/mL fibronectin was added to each well of a suspension six-well plate, and the suspension six-well plate was coated overnight at 4° C., and washed twice with PBS to remove unbound proteins. 2×10⁵ human peripheral blood-derived T cells (hT cells) were added to each well, and 1 mL of ImmunoCult TM-XF T cell medium containing 8 μg/mL polypropylene and 10 ng/mL IL-2 was added. Then, 10⁷ IU of lentivirus-packaged plasmids encoding huGD2.CD28.4-1BB.z-CAR-GFP were added, and the plate was centrifuged at 1500 g for 60 min, once every 8 h, for a total of 3 times. Thereafter, the transfection medium was replaced with 2 mL of fresh T cell medium to continue culture expansion. When the positive expression rate of CAR protein of CAR-T cells was greater than 30%, the culture expansion was continued for subsequent studies. The density of the prepared CAR-T cell suspension was adjusted to 1×10⁶ cells/mL. A certain amount of cell membrane anchoring molecules (DSPE-PEGₖ₅-Tre) were added to each milliliter of cell suspension, and the cell suspension was incubated at 4° C. for 30 min, and centrifuged at 1500 rmp for 5 min. The supernatant was discarded, and cells were washed 2-3 times with PBS and resuspended to obtain CAR-T cells with reactive groups on the surface. The nano-drug BCN-Ava-Lip was adjusted to isotonic and diluted into a solution with an avasimibe concentration of 150 μg/mL. The solution was incubated with the CAR-T cells with active reactive groups on the surface at 25° C. for 20 min, and centrifuged at 1500 rmp for 5 min. The supernatant was discarded, and cells were washed with PBS to remove the unreacted nano-drug, and resuspended to obtain CAR-T cells modified with the nano-drug on the surface, namely a BCN-Ava-Lip/CAR-T cell drug.

### EMBODIMENT 15

Preparation of CAR-T cell drug (ADIBO-PTX-NPs/CAR-T cell) CAR-T cells were prepared according to the method of embodiment 14. The density of the prepared CAR-T cell suspension was adjusted to 1×10⁶ cells/mL. A certain amount of cell membrane anchoring molecules (SA₂-Glu-PEG₁ₖ-N₃) were added to each milliliter of cell suspension, and the cell suspension was incubated at 4° C. for 20 min, and centrifuged at 1500 rmp for 5 min. The supernatant was discarded, and cells were washed 2-3 times with PBS and resuspended to obtain CAR-T cells with reactive groups on the surface. The nano-drug (ADIBO-PTX-NPs) was adjusted to isotonic and diluted into a solution with a paclitaxel concentration of 100 μg/mL. The solution was incubated with the CAR-T cells with active reactive groups on the surface at 37° C. for 45 min, and centrifuged at 1500 rmp for 5 min. The supernatant was discarded, and cells were washed with PBS to remove the unreacted nano-drug, and resuspended to obtain CAR-T cells modified with the nano-drug on the surface, namely an ADIBO-PTX-NPs/CAR-T cell drug.

### EMBODIMENT 16

Preparation of murine T cell drug (BCN-Ava-Lip/mT cells)

The density of a mouse spleen-derived T cell (mT cell) suspension was adjusted to 1×10⁶ cells/mL. A certain amount of cell membrane anchoring molecules (DSPE-PEG₅ₖ-Tre) were added to each milliliter of cell suspension, and the cell suspension was incubated at 4° C. for 30 min, and centrifuged at 1500 rmp for 5 min. The supernatant was discarded, and cells were washed 2-3 times with PBS and resuspended to obtain mT cells with reactive groups on the surface. The nano-drug (BCN-Ava-Lip) was adjusted to isotonic and diluted into a solution with an avasimibe concentration of 150 μg/mL. The solution was incubated with the mT cells with active reactive groups on the surface at 25° C. for 20 min, and centrifuged at 1500 rmp for 5 min. The supernatant was discarded, and cells were washed with PBS to remove the unreacted nano-drug, and resuspended to obtain murine T cells modified with the nano-drug on the surface, namely a BCN-Ava-Lip/mT cell drug.

EMBODIMENT 17

Preparation of TCR-T cell drug (BCN-Ava-Lip/TCR-T cell)

The density of a Pmel-1 or OT-1 mouse spleen-derived T cell (TCR-T cell) suspension was adjusted to 1×10⁶ cells/mL. A certain amount of cell membrane anchoring molecules (DSPE-PEG 5k -Tre) were added to each milliliter of cell suspension, and the cell suspension was incubated at 4° C. for 30 min, and centrifuged at 1500 rmp for 5 min. The supernatant was discarded, and cells were washed 2-3 times with PBS and resuspended to obtain TCR-T cells with reactive groups on the surface. The nano-drug (BCN-Ava-Lip) was adjusted to isotonic and diluted into a solution with an avasimibe concentration of 150 μg/mL. The solution was incubated with the TCR-T cells with active reactive groups on the surface at 25° C. for 20 min, and centrifuged at 1500 rmp for 5 min. The supernatant was discarded, and cells were washed with PBS to remove the unreacted nano-drug, and resuspended to obtain TCR-T cells modified with the nano-drug on the surface, namely a BCN-Ava-Lip/TCR-T cell drug.

### EMBODIMENT 18

Preparation of human neutrophil drug (BCN-Ava-Lip/hNEs)

The density of a human peripheral blood-derived neutrophil (hNEs) suspension was adjusted to 1×10⁶ cells/mL. A certain amount of cell membrane anchoring molecules (DSPE-PEG₅ₖ-Tre) were added to each milliliter of cell suspension, and the cell suspension was incubated at 4° C. for 30 min, and centrifuged at 1500 rmp for 5 min. The supernatant was discarded, and cells were washed 2-3 times with PBS and resuspended to obtain hNEs with reactive groups on the surface. The nano-drug (BCN-Ava-Lip) was adjusted to isotonic and diluted into a solution with an avasimibe concentration of 150 μg/mL. The solution was incubated with the hNEs with active reactive groups on the surface at 25° C. for 20 min, and centrifuged at 1500 rmp for 5 min. The supernatant was discarded, and cells were washed with PBS to remove the unreacted nano-drug, and resuspended to obtain human neutrophils modified with the nano-drug on the surface, namely a BCN-Ava-Lip/hNEs cell drug.

### EMBODIMENT 19

Preparation of human neutrophil drug (Mal-siRNA-Lip/hNEs)

The density of a human peripheral blood-derived neutrophil (hNEs) suspension was adjusted to 1×10⁶ cells/mL. A certain amount of cell membrane anchoring molecules (DOPE-PEG₂ₖ-SH) were added to each milliliter of cell suspension, and the cell suspension was incubated at 4° C. for 15 min, and centrifuged at 1500 rmp for 5 min. The supernatant was discarded, and cells were washed 2-3 times with PBS and resuspended to obtain hNEs with reactive groups on the surface. The nano-drug (Mal-siRNA-Lip) was adjusted to isotonic and diluted into a solution with an siRNA concentration of 200 nM. The solution was incubated with the hNEs with reactive groups on the surface at 4° C. for 2 h, and centrifuged at 1500 rmp for 5 min. The supernatant was discarded, and cells were washed with PBS to remove the unreacted nano-drug, and resuspended to obtain human neutrophils modified with the nano-drug on the surface, namely a Mal-siRNA-Lip/hNEs cell drug.

### EMBODIMENT 20

Preparation of murine neutrophil drug (BCN-Ava-Lip/mNEs)

The density of a mouse bone marrow-derived neutrophil (mNEs) suspension was adjusted to 1×10⁶ cells/mL. A certain amount of cell membrane anchoring molecules (DSPE-PEG₅ₖ-Tre) were added to each milliliter of cell suspension, and the cell suspension was incubated at 4° C. for 30 min, and centrifuged at 1500 rmp for 5 min. The supernatant was discarded, and cells were washed 2-3 times with PBS and resuspended to obtain mNEs with reactive groups on the surface. The nano-drug (BCN-Ava-Lip) was adjusted to isotonic and diluted into a solution with an avasimibe concentration of 150 μg/mL. The solution was incubated with the mNEs with active reactive groups on the surface at 25° C. for 20 min, and centrifuged at 1500 rmp for 5 min. The supernatant was discarded, and cells were washed with PBS to remove the unreacted nano-drug, and resuspended to obtain murine neutrophils modified with the nano-drug on the surface, namely a BCN-Ava-Lip/mNEs cell drug.

### EMBODIMENT 21

Preparation of human mesenchymal stem cell drug (ADIBO-PTX-NPs/hMSC)

The density of a human umbilical cord-derived mesenchymal stem cell (hMSC cell) suspension was adjusted to 1×10⁶ cells/mL. A certain amount of cell membrane anchoring molecules (SA₂-Glu-PEG₁ₖ-N₃) were added to each milliliter of cell suspension, and the cell suspension was incubated at 4° C. for 20 min, and centrifuged at 1500 rmp for 5 min. The supernatant was discarded, and cells were washed 2-3 times with PBS and resuspended to obtain hMSC cells with reactive groups on the surface. The nano-drug (ADIBO-PTX-NPs) was adjusted to isotonic and diluted into a solution with a paclitaxel concentration of 100 μg/mL. The solution was incubated with the hMSC cells with active reactive groups on the surface at 37° C. for 45 min, and centrifuged at 1500 rmp for 5 min. The supernatant was discarded, and cells were washed with PBS to remove the unreacted nano-drug, and resuspended to obtain human MSC cells modified with the nano-drug on the surface, namely an ADIBO-PTX-NPs/hMSC cell drug.

### EMBODIMENT 22

Preparation of tumor cells (BCN-Ava-Lip/A549 cells)

The density of a lung cancer cell A549 suspension was adjusted to 1×10⁶ cells/mL. A certain amount of cell membrane anchoring molecules (DSPE-PEG₅ₖ-Tre) were added to each milliliter of cell suspension, and the cell suspension was incubated at 4° C. for 30 min, and centrifuged at 1500 rmp for 5 min. The supernatant was discarded, and cells were washed 2-3 times with PBS and resuspended to obtain A549 cells with reactive groups on the surface. The nano-drug (BCN-Ava-Lip) was adjusted to isotonic and diluted into a solution with an avasimibe concentration of 150 μg/mL. The solution was incubated with the A549 cells with active reactive groups on the surface at 25° C. for 20 min, and centrifuged at 1500 rmp for 5 min. The supernatant was discarded, and cells were washed with PBS to remove the unreacted nano-drug, and resuspended to obtain tumor cells modified with the nano-drug on the surface, namely BCN-Ava-Lip/A549 cells.

### EMBODIMENT 23

Determination of cell drug loading

Eleven different cell drugs prepared in Embodiments 12-22 were centrifuged at 1500 rmp for 5 min, the supernatant was discarded, an appropriate volume of SDS cell lysate was added to the cell pellet, and the mixture was fully mixed by vortex and allowed to stand at 4° C. for 30 min. 4 times of acetonitrile by volume was added for carrying out protein precipitation and drug extraction, the mixture was allowed to stand at 4° C. for 30 min, mixed by vortex at 1500 rpm for 5 min, and centrifuged at 12000 rpm for 10 min, and the supernatant was obtained for HPLC or microplate detection. The results showed that the drug loadings of the BCN-Ava-Lip/hT cells, the BCN-Ava-Lip/CAR-T cells, the BCN-Ava-Lip/mT cells, the BCN-Ava-Lip/TCR-T cells, the BCN-Ava-Lip/hNEs, the BCN-Ava-Lip/mNEs, the ADIBO-PTX-NPs/hT cells, the ADIBO-PTX-NPs/CAR-T cells, the Mal-siRNA-Lip/hNEs, the ADIBO-PTX-NPs/hMSC, and the BCN-Ava-Lip/A549 cells were 4.92 1.μg Ava/10⁶ hT cells, 4.65 1.μg Ava/10⁶ CAR-T cells, 4.38 1.tg Ava/10⁶ mT cells, 4.42 1.μg Ava/10⁶ TCR-T cells, 4.14 μg Ava/10⁶ hNEs, 3.61 μg Ava/10⁶ mNEs, 10.82 μg PTX/10⁶ hT cells, 8.25 μg PTX/10⁶ CAR-T cells, 83 nM siRNA/10⁶ hNEs, 8.36 μg PTX/10⁶ hMSC cells, and 6.95 μg Ava/10⁶ A549 cells respectively.

### EMBODIMENT 24

Laser confocal characterization of cell drug

100 mg of SPC, 15 mg of cholesterol and 25 mg of DSPE-BCN were dissolved in chloroform and methanol, and rhodamine B-1,2-dihexadecyl-3-glycero-phosphoethanolamine triethylammonium salt (RhoB-DHPE) (2 mg/mL, 25 μL) was added. The organic solvent was removed by rotary evaporation. The reaction solution was dried in vacuum overnight, hydrated at 37° C. for 30 min, sonicated with a probe for 10-30 min, and then passed through 0.80, 0.45, and 0.22 1.tm filter membranes in turn to obtain a fluorescently-labeled nano-drug RhoB-BCN-Lip. According to the preparation method of the above cell drug, the surfaces of different cells were modified with the fluorescently-labeled nano-drug RhoB-BCN-Lip to obtain five fluorescently-labeled cell drugs (RhoB-BCN-Lip/mT cells, RhoB-BCN-Lip/hT cells, RhoB-BCN-Lip/CAR-T cells, RhoB-BCN-Lip/mNEs, and RhoB-BCN-Lip/hNEs).

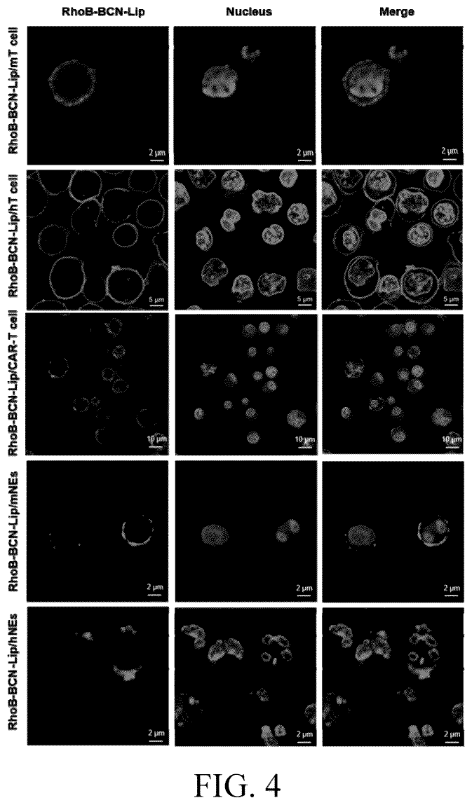

The freshly prepared fluorescently-labeled cell drugs were fluorescently labeled with a nuclear dye Hoechst33342 (1 μg/mL), fixed with paraformaldehyde (PFA), and then photographed by confocal laser (FIG. 4). From the figure, red fluorescence of rhodamine exists on the cell membrane, which indicates that the living cells are successfully modified with the fluorescently-labeled nano-drug by the anchoring modification technology on the surface of a living cell disclosed in the present invention.

### EMBODIMENT 25

Detection of cell drug viability

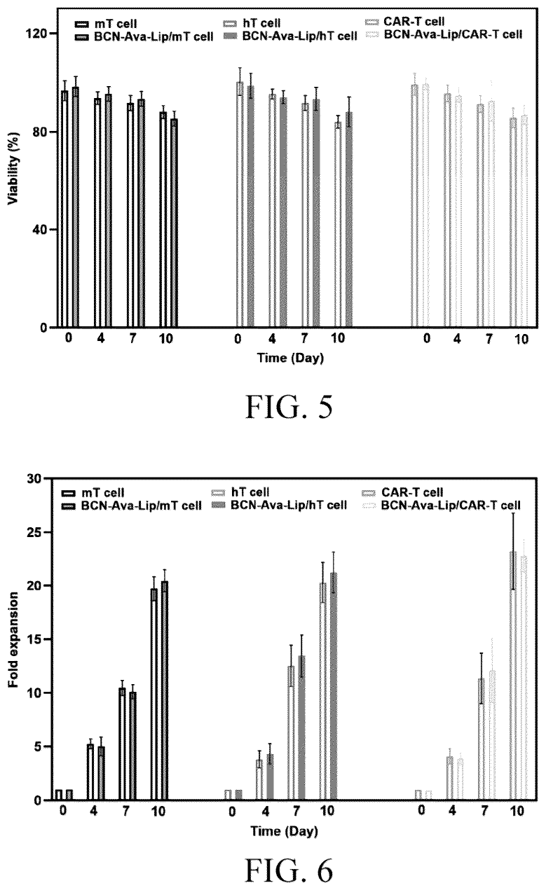

Taking murine T cells as an example, BCN-Ava-Lip/mT cells were prepared according to the method of Embodiment 16. Afterwards, the BCN-Ava-Lip/mT cells were cultured and expanded in a medium containing 5 μg/mL anti-CD3 antibodies, 2 μg/mL anti-CD28 antibodies and 10 ng/mL interleukin-2 (IL-2). On the 0th, 4th, 7th, and 10th days of culture expansion, the cells were stained with trypan blue, and counted under an inverted fluorescence microscope, and the viabilities of the cells in the expansion process were calculated. Expanded and cultured mT cells were used as a positive control. Viability =unstained cells/total cells×100%. The detection method of the viability of the human T cell drug BCN-Ava-Lip/hT cells and the CAR-T cell drug BCN-Ava-Lip/CAR-T cells is the same as that of the BCN-Ava-Lip/mT cells. The results of viability detection are shown in FIG. 5. The results show that the viability of the cell drug group has no significant difference from that of the positive control group, and the cell viabilities are both greater than 80%, which indicates that the cell drug prepared by the anchoring modification technology on the surface of a living cell disclosed in the present invention cannot affect the cell viability.

### EMBODIMENT 26

Characterization of proliferation ability of cell drug

Taking murine T cells as an example, BCN-Ava-Lip/mT cells were prepared according to the method of Embodiment 16. Afterwards, the BCN-Ava-Lip/mT cells were cultured and expanded in a medium containing 5 μg/mL anti-CD3 antibodies, 2 μg/mL anti-CD28 antibodies and 10 ng/mL interleukin-2 (IL-2). On the 0th, 4th, 7th, and 10th days of culture expansion, the cells were counted. Expanded and cultured mT cells were used as a control. In vitro expansion fold of cells=number of cells after stimulation/number of cells before stimulation. The proliferation characterization method of the BCN-Ava-Lip/hT cells and the BCN-Ava-Lip/CAR-T cells is the same as that of the BCN-Ava-Lip/mT cells. The proliferation ability is shown in FIG. 6. The results show that the proliferation ability of the cell drug group has no significant difference from that of the positive control group, which indicates that the cell drug prepared by the anchoring modification technology on the surface of a living cell disclosed in the present invention cannot affect the cell proliferation ability.

### EMBODIMENT 27

Characterization of chemotactic ability of cell drug

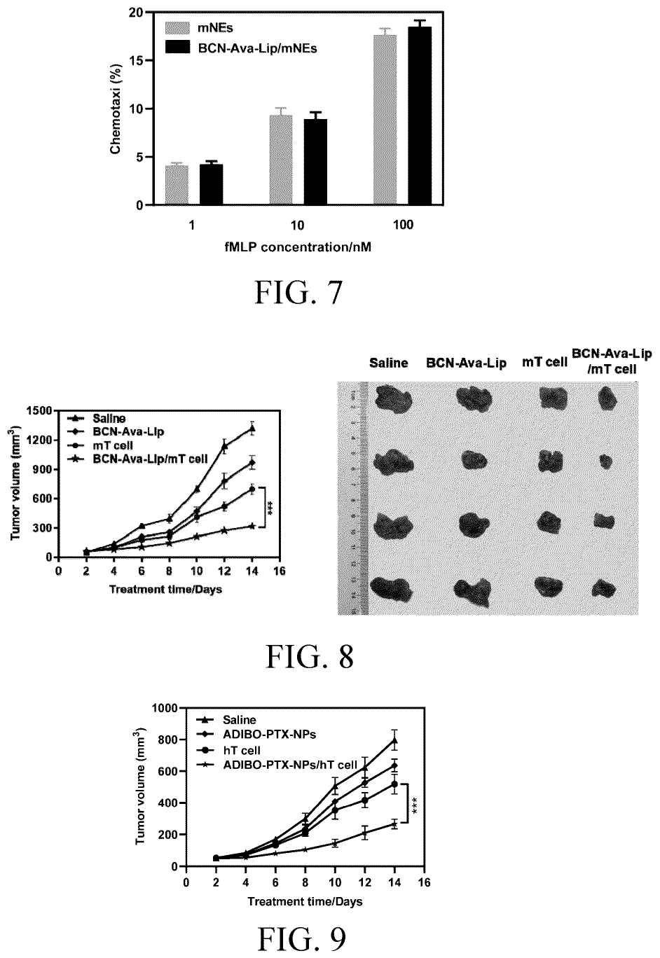

Taking murine neutrophils as an example, BCN-Ava-Lip/mNEs were prepared according to the method of Embodiment 17. 1×10⁶ BCN-Ava-Lip/mNEs were plated in an upper chamber of a Transwell dish, and media containing chemotactic tripeptides (fMLP) with the final concentrations of 1 nM, 10 nM and 100 nM were added to lower chambers. After incubation in 5% CO₂ at 37° C. for 12 h, the dish was taken out, and cells in the upper chamber and chemotactic cells in the lower chamber were collected respectively and counted to calculate the chemotactic index. A medium without fMLP was added to a lower chamber as a blank control, and other operations were the same. mNEs were added to an upper chamber, and media containing fMLP with the final concentrations of 1 nM, 10 nM and 100 nM were added to lower chambers as a positive control group, and other operations were the same. Chemotactic index =(number of cells in the lower chamber of the experimental group—number of cells in the lower chamber of the blank control group)/total number of cells. The results of chemotactic ability are shown in FIG. 7. The results show that the chemotactic ability of the cell drug group has no significant difference from that of the positive control group, which indicates that the cell drug prepared by the anchoring modification technology on the surface of a living cell disclosed in the present invention cannot affect the cell chemotactic ability.

### EMBODIMENT 28

Tumor treatment effect of cell drug (BCN-Ava-Lip/mT cells)

Taking the inhibitory effect of the murine T cell drug BCN-Ava-Lip/mT cells on melanoma as an example, 16 C57BL/6J mice were intradermally inoculated with a Bl6F10 melanoma cell suspension as per 2×10⁶ cells/mouse on the right back to establish in situ melanoma models. After inoculation, the mice were kept in a clean rearing room, given sufficient water and feed, and observed the tumor growth every day. The diameters of the tumor were measured with a vernier caliper, and the tumor volume was calculated according to the formula V=L×W×W/2, where L is the long diameter of the tumor, and W is the short diameter of the tumor. When the tumor volume in the C57BL/6J mice reached 50 mm³, the mice were randomly divided into 4 groups with 4 mice in each, and were respectively given: 1) physiological saline; 2) BCN-Ava-Lip (Ava: 2 mg/kg); 3) mT cells (1×10⁷ cells/mouse); and 4) BCN-Ava-Lip/mT cells (1×10⁷ cells/mouse, Ava: 2 mg/kg). The first administration was recorded as on day 0, and intratumoral injection was carried out on days 0, 3, 6, 9 and 12 respectively, for a total of 5 administrations. From day 0 of administration, the long and short diameters of the tumor were measured every other day, and the tumor volume was calculated. The time (day) was used as the abscissa and the tumor volume (mm 3) was the ordinate to draw a tumor growth curve. On day 14 after administration, the tumor-bearing mice were euthanized, the tumor tissue was carefully separated, the tumors were photographed, and the tumor size was observed, as shown in FIG. 8. The results show that compared with the T cell group and the nano-drug group (BCN-Ava-Lip), the cell drug group (BCN-Ava-Lip/mT cells) has the best antitumor effect.

### EMBODIMENT 29

Tumor treatment effect of cell drug (ADIBO-PTX-NPs/hT cells)

Taking the inhibitory effect of the human T cell drug ADIBO-PTX-NPs/hT cells on breast cancer as an example, 20 BALB/c mice were inoculated with a human breast cancer cell (4T1 breast cancer cell) suspension as per 3×10⁶ cell/mouse in the right mammary fat pad to establish orthotopic breast cancer models. After inoculation, the mice were kept in a clean rearing room, given sufficient water and feed, and observed the tumor growth every day. The diameters of the tumor were measured with a vernier caliper, and the tumor volume was calculated according to the formula V=L×W×W/2, where L is the long diameter of the tumor, and W is the short diameter of the tumor. When the tumor volume in the BALB/c mice reached 50 mm³, the mice were randomly divided into 4 groups with 5 mice in each, and were respectively given: 1) physiological saline; 2) ADIBO-PTX-NPs (PTX: 5 mg/kg); 3) hT cells (1×10⁷ cells/mouse); and 4) ADIBO-PTX-NPs/hT cells (1×10⁷ cells/mouse, PTX: 5 mg/kg). The first administration was recorded as on day 0, and intravenous injection was carried out on days 0, 6 and 12 respectively, for a total of 3 administrations. From day 0 of administration, the long and short diameters of the tumor were measured every other day, and the tumor volume was calculated. The time (day) was used as the abscissa and the tumor volume (mm³) was the ordinate to draw a tumor growth curve, as shown in FIG. 9. The results show that compared with the T cell group and the nano-drug group (ADIBO-PTX-NPs), the cell drug group (ADIBO-PTX-NPs/hT cells) has the best antitumor effect.

### EMBODIMENT 30

Tumor treatment effect of cell drug (BCN-Ava-Lip/CAR-T cells)

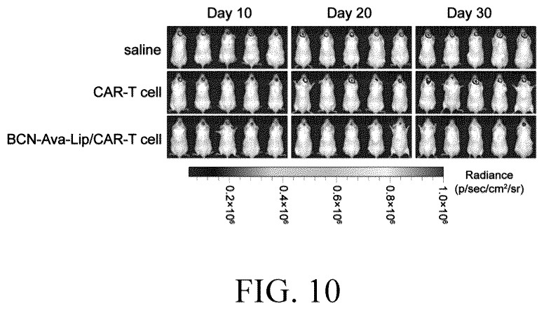

Taking the inhibitory effect of the CAR-T cell drug BCN-Ava-Lip/CAR-T cells on glioma as an example, 15 severely immunodeficient mice (NSG mice) were inoculated with a human glioma cell (LN229 glioma cell) suspension as per 2×10⁵ cells/mouse in the brain to establish orthotopic glioma models. After inoculation, the mice were given sufficient water and feed, and the tumor growth was observed by in vivo imaging. When the NSG mouse glioma models were successfully established, the mice were randomly divided into 3 groups with 5 mice in each, and were respectively given: 1) physiological saline; 2) CAR-T cells (5×10⁶ cells/mouse); and 3) BCN-Ava-Lip/CAR-T cells (5×10⁶ cells/mouse, Ava: 1 mg/kg). The first administration was recorded as on day 0, and in situ injection was carried out in the brain on days 0, 6 and 12 respectively, for a total of 3 administrations. From day 0 of administration, the tumor growth in the mice was observed by in vivo imaging, as shown in FIG. 10. The results show that compared with the CAR-T cell group, the cell drug group (BCN-Ava-Lip/CAR-T cells) has the best antitumor effect.

1. A method for anchoring and modifying a nano-drug on the surface of a cell, comprising: introducing an active reactive group to the surface of a living cell by means of a hydrophobic tail chain of a cell membrane anchoring molecule; modifying the surface of a nano-drug with a corresponding reactive group; and carrying out a biological orthogonal click reaction between the active reactive group of the cell membrane anchoring molecule modified on the surface of the living cell and the corresponding reactive group modified on the surface of the nano-drug, so as to anchor and modify the nano-drug to the surface of the cell to obtain a living cell modified with the nano-drug.

2. The method according to claim 1, wherein the structural general formula of the cell membrane anchoring molecule is as follows: 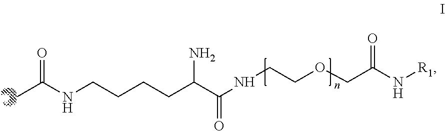
wherein R₁ is a common lipid or a long-chain alkane of 6-20 C; [symbol]
is an active reactive group, which is any one selected from a group consisting of azide, azadibenzocyclooctyne, sulfydryl, amino, maleimide, α,β-unsaturated carbonyl, tetrazine, and bicyclo[6.1.0]nonyne.

3. The method according to claim 2, wherein the common lipid is selected from a group consisting of distearoyl phosphatidyl ethanolamine, dioleoyl phosphatidyl ethanolamine, 1,2-dihexadecyl-3-glycero-phosphoethanolamine or cholesterol.

4. The method according to claim 2, wherein the n=20-100.

5. The method according to claim 2, wherein the active reactive group [symbol] is any one selected from a group consisting of tetrazine, bicyclo[6.1.0]nonyne, azide, and azadibenzocyclooctyne.

6. The method according to claim 2, wherein the living cells are selected from primary cells or immortalized cells with lipid membrane structures of humans or animals.

7. The method according to claim 2, wherein the nano-drug is nanoparticles loaded with a therapeutic agent; and the nanoparticles are liposomes, nanovesicles, solid lipid nanoparticles or micelles with a particle size of 1-1000 nm.

8. The method according to claim 7, wherein the therapeutic agent drugs.

9. The method according to claim 8, wherein the therapeutic agent is selected from avasimibe, paclitaxel or PD-1 monoclonal antibodies.

10. The method according to claim 2, wherein the corresponding reactive group is modified to the surface of the nano-drug by a corresponding reactive group modifier; the corresponding reactive group modifier is a lipid containing a corresponding reactive group, having a general formula as follows: 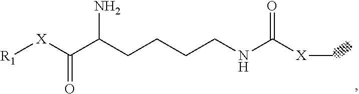
wherein X═—NH, O,
R₁ is a common lipid or a long-chain alkane with a chain length of 6-20 C; and [symbol]
is the corresponding reactive group, which is any one selected from a group consisting of azadibenzocyclooctyne, azide, maleimide, sulfydryl, amino, bicyclo[6.1.0]nonyne, and tetrazine.

11. The method according to claim 1, wherein the biological orthogonal click reaction is selected from ketone/hydroxylamine condensation, Michael addition of a sulfydryl or amino group with maleimide, strain-promoted azide-alkyne cycloaddition, or strain-promoted inverse electron-demand Diels—Alder cycloaddition.

12. The method according to claim 1, wherein the cell membrane anchoring molecule is incubated with a living cell at 0-40° C. for 5-120 min to obtain a living cell modified with the cell membrane anchoring molecule on the surface; and the nano-drug modified with the corresponding reactive group on the surface is incubated with the living cell modified with the cell membrane anchoring molecule on the surface at 0-37° C. for 5-120 min to obtain a living cell modified with the nano-drug.

13. A living cell modified with a nano-drug prepared by the method according to claim 1.

14. An application of the living cell modified with a nano-drug according to claim 13 in preparation of a drug for treating tumors or inflammation-related diseases.

15. The application according to claim 14, wherein the tumors comprise melanoma, glioma, breast cancer or ovarian cancer; and the inflammation-related diseases is selected stroke or arthritis.

16. A cell membrane anchoring molecule, having a general formula as follows: 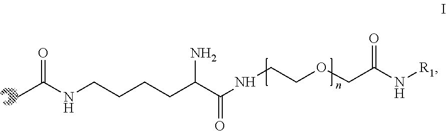
wherein R₁ is a common lipid or a long-chain alkane of 6-20 C; [symbol]
is an active reactive group, which is any one selected from a group consisting of azide, azadibenzocyclooctyne, sulfydryl, amino, maleimide, a,(3-unsaturated carbonyl, tetrazine, and bicyclo[6.1.0]nonyne.

17. The cell membrane anchoring molecule according to claim 16, wherein the common lipid is selected from a group consisting of distearoyl phosphatidyl ethanolamine, dioleoyl phosphatidyl ethanolamine, 1,2-dihexadecyl-3-glycero-phosphoethanolamine, and cholesterol; the n=20-100; and the active reactive group [symbol] is any one selected from a group consisting of tetrazine, bicyclo[6.1.0]nonyne, azide, and azadibenzocyclooctyne.

18. A method for synthesizing the cell membrane anchoring molecule according to claim 16, comprising the following steps:

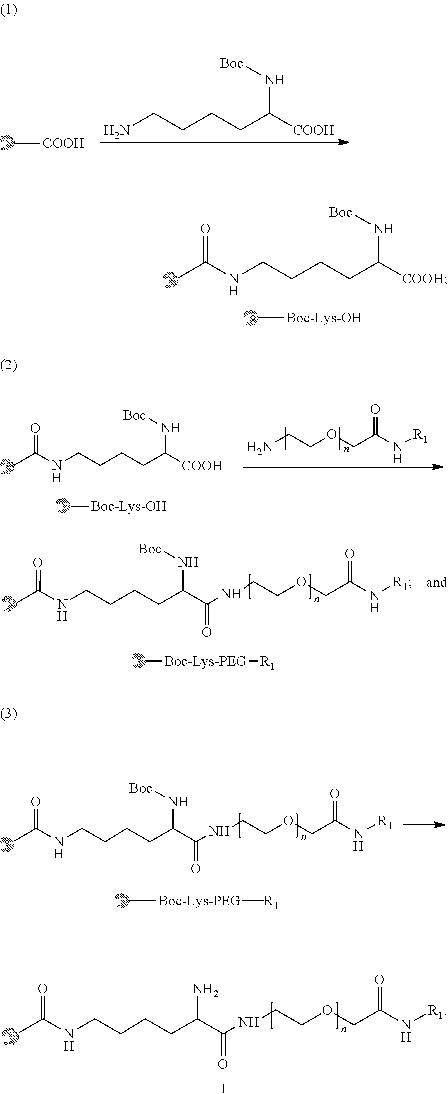

19. An application of the cell membrane anchoring molecule according to claim 16 in preparation of a living cell drug, wherein the living cell drug is a living cell modified with a nano-drug on the surface.

20. A corresponding reactive group modifier, having a general formula as follows: 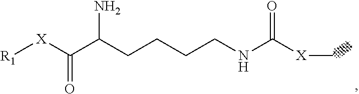
wherein X═—NH, O,
R₁ is a common lipid or a long-chain alkane with a chain length of 6-20 C; and [symbol] is the corresponding reactive group, which is any one selected from a group consisting of azadibenzocyclooctyne, azide, maleimide, sulfydryl, amino, bicyclo[6.1.0]nonyne, and tetrazine.

21. The corresponding reactive group modifier according to claim 20, wherein the common lipid is selected from a group consisting of distearoyl phosphatidyl ethanolamine, dioleoyl phosphatidyl ethanolamine, 1,2-dihexadec y1-3 -glycero-phosphoethanolamine, and cholesterol.

22. A method for synthesizing the corresponding reactive group modifier according to claim 20, comprising the following steps:

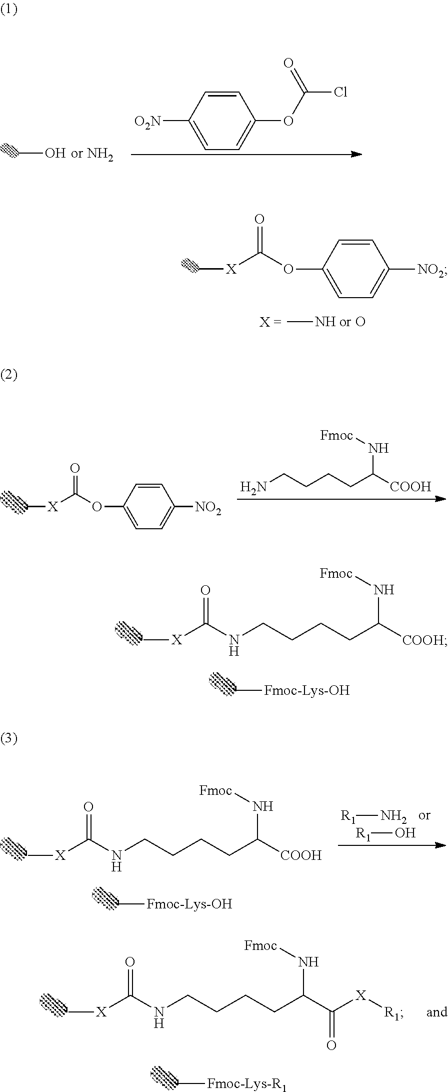

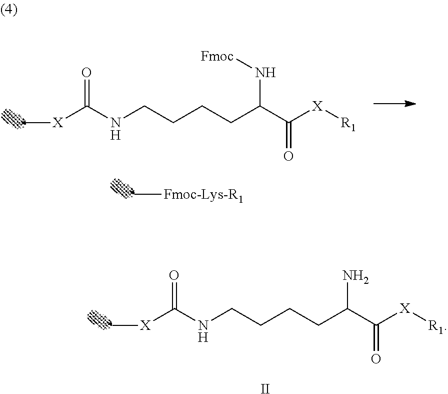

23. An application of the corresponding reactive group modifier according to claim 20 in preparation of a living cell drug, wherein the living cell drug is a living cell modified with a nano-drug on the surface.
